(19) United States
(12) Patent Application Publication
(10) Pub. No.: US 2013/0022989 A1
(43) Pub. Date: Jan. 24, 2013

(54) DENTAL STEM CELL REPROGRAMMING

(71) Applicants: Jeremy J. Mao, Closter, NJ (US); Mo Chen, Astoria, NY (US)

(72) Inventors: Jeremy J. Mao, Closter, NJ (US); Mo Chen, Astoria, NY (US)

(73) Assignee: The Trustees of Columbia University in the City of New York, New York, NY (US)

(21) Appl. No.: 13/381,063

(22) PCT Filed: Jun. 25, 2010
(86) PCT No.: PCT/US10/39929

§ 371 Date: Aug. 13, 2012

Related U.S. Application Data
(60) Provisional application No. 61/220,368, filed on Jun. 25, 2009.
(60) Provisional application No. 61/291,796, filed on Dec. 31, 2009.
(60) Provisional application No. 61/327,261, filed on Apr. 23, 2010.

(51) Int. Cl. C12N 15/867; C12Q 1/04; G01N 33/566; C12N 5/10

(52) U.S. Cl. 435/7.1; 435/325; 435/371; 435/456; 435/34

(57) ABSTRACT

Provided is dental stem cell comprising an Oct3/4 transgene. Also provided is a method of making a pluripotent stem cell. Additionally, a method of preparing an insulin-secreting cell is provided. Further provided is an insulin-secreting cell prepared by that method. A method of preparing a chondrocyte-like cell is also provided, as is a chondrocyte-like cell prepared by that method. Additionally provided is a method of preparing a myocyte-like cell. Also, a myocyte-like cell prepared by that method is provided. A method of preparing a hair follicle-like cell is additionally provided, as is a hair follicle-like cell prepared by that method. A method of preparing a neuron-like cell is additionally provided, as is a neuron-like cell prepared by that method.

### CROSS-REFERENCE TO RELATED APPLICATIONS

The present application claims the benefit of U.S. Provisional Application No. 61/220,368 filed 25 Jun. 2009, U.S. Provisional Application No. 61/291,796 filed 31 Dec. 2009, and U.S. Provisional Application No. 61/327,261 filed 23 Apr. 2010; which are incorporated herein by reference in their entireties.

### STATEMENT REGARDING FEDERALLY SPONSORED RESEARCH OR DEVELOPMENT

Not Applicable.

### MATERIAL INCORPORATED-BY-REFERENCE

Not Applicable.

### FIELD OF THE DISCLOSURE

The present application generally relates to dental stem cells. More specifically, this application relates to pluripotent cells derived from dental stem cells and cells further differentiated therefrom.

### BACKGROUND

Stem cells have become the centerpiece of regenerative medicine (Alhadlaq and Mao, 2004; Marion and Mao, 2006). Different types of stem cells include embryonic stem cells, amniotic fluid stem cells, umbilical cord stem cells, and adult stem cells from bone marrow, skeletal muscle and adipose tissue (Mao et al., 2007). Somatic cells can be reprogrammed into induced pluripotent stem cells (iPS) by transfecting the somatic cells with genes for the four factors Oct3/4, Sox2, Nanog and Lin28 (Yu et al., 2007; U.S. Patent Application Publication US20090047263). Expression of native and transgenic Oct3/4 and other factors has also been evaluated in mesenchymal stem cells (Liu et al., 2008; Meuller et al., 2009; PCT Patent Application PCT/US09/39360).

The tooth functions to process food, and also is important for aesthetics, vocal communicating, including speech in humans, and digestion. Tooth pulp is neural crest-derived mesenchymal tissue, and its genesis relies on epithelial-mesenchymal interactions. Dental-pulp stem/progenitor cells, or “dental stem cells” (DSCs) express the embryonic stem cell markers Nanog and Oct3/4, suggesting their primitive status. These cells from the tooth can differentiate into osteoblasts, neuron-like cells and adipocytes (Miura et al., 2003; U.S. Patent Publication US20070274958A1). See also PCT Patent Publications WO04073633A2, WO03066840A2, WO07014639A2, WO06010600A2 and WO0207679A2 and PCT Patent Application PCT/US09/39360.

Insulin-producing cells (IPCs) have been derived from embryonic stem cells and postnatal stem cells isolated from anatomic structures such as amniotic fluid, bone marrow, and adipose tissue (D'Amour et al, 2006; Lumelsky et al., 2001). A common challenge for this task is insulin yield.

### SUMMARY

In some embodiments, a dental stem cell comprising an Oct3/4 transgene is provided.

In other embodiments, a method of making a pluripotent stem cell is provided. The method comprises transfecting a dental stem cell with an Oct3/4 gene such that the cell expresses a transgenic Oct3/4 and is pluripotent. In some embodiments, the dental stem cell is transfected with an Oct3/4 gene and at least one of a Nanog transgene, a Sox2 transgene or a Lin28 transgene. In some embodiments, the dental stem cell is transfected with an Oct3/4 gene and a Sox2 transgene.

Also provided is a method of preparing an insulin-secreting cell. The method comprises incubating a pluripotent dental stem cell expressing an Oct3/4 transgene in a medium that induces differentiation of a pluripotent cell into an insulin-secreting cell, under conditions such that the dental stem cell differentiates into an insulin-secreting cell. An insulin-secreting cell prepared by this method is also provided.

Additionally, a method of preparing a chondrocyte-like cell is provided. The method comprises incubating a pluripotent dental stem cell expressing an Oct3/4 transgene in a medium that induces differentiation of a pluripotent cell into a chondrocyte-like cell, under conditions such that the dental stem cell differentiates into a chondrocyte-like cell. A chondrocyte-like cell prepared by this method is also provided.

In further embodiments, a method of preparing a myocyte-like cell is provided. The method comprises incubating a pluripotent dental stem cell expressing an Oct3/4 transgene in a medium that induces differentiation of a pluripotent cell into a myocyte-like cell, under conditions such that the dental stem cell differentiates into a myocyte-like cell. A myocyte-like cell prepared by this method is also provided.

In additional embodiments, a method of preparing a hair follicle-like cell is provided. The method comprises incubating a pluripotent dental stem cell expressing an Oct3/4 transgene in a medium that induces differentiation of a pluripotent cell into a hair follicle-like cell, under conditions such that the dental stem cell differentiates into a hair follicle-like cell. A hair follicle-like cell prepared by this method is also provided.

In additional embodiments, a method of preparing a neuron-like cell is provided. The method comprises incubating a pluripotent dental stem cell expressing an Oct3/4 transgene and a Sox2 transgene in a medium that induces differentiation of a pluripotent cell into a neuron-like cell, under conditions such that the dental stem cell differentiates into a neuron-like cell. A neuron-like cell prepared by this method is also provided.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a timeline of an experimental protocol.

FIG. 2 is a micrograph of an induced pluripotent stem (iPS) cell colony twelve days after seeding onto media.

FIG. 3 is micrographs of colonies formed by dental stem cells expressing transgenic Oct3/4 (O), Nanog (N), Sox2 (S), and/or Lin28 (L) as labeled, twelve days after transfection with the transgene(s) on a lentiviral vector.

FIG. 4 is micrographs of colonies formed by dental stem cells expressing transgenic Oct3/4 (O), Nanog (N), Sox2 (S), and/or Lin28 (L) as labeled, three weeks after transfection with the transgene(s) on a lentiviral vector.

FIG. 5 is micrographs of colonies formed by dental stem cells expressing transgenic Oct3/4 (O), Nanog (N), Sox2 (S), and/or Lin28 (L) as labeled, showing continued expansion of the colonies after transfer to new tissue culture dishes more than three weeks after transfection with the transgene(s) on a lentiviral vector.

FIG. 6 shows first passage (P1) and second passage (P2) dental pluripotent stem cell (DPSC) colonies transfected with the Oct3/4 gene.

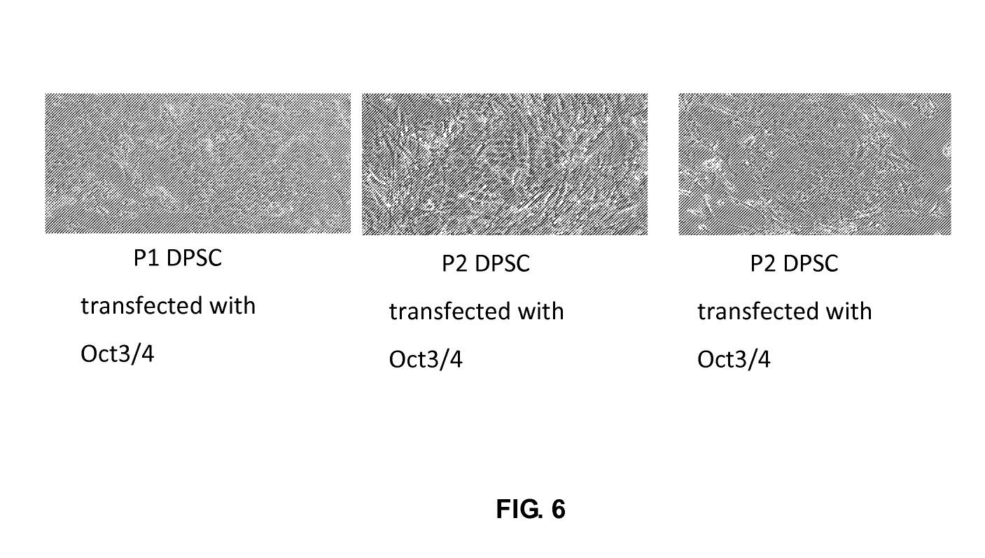

FIG. 7 shows phase contrast (PH) and DAPI filtered fluorescence (DAPI) views of Oct3/4 transfected colonies treated with an antibody to Nanog or Oct3/4. The colonies were also tested for alkaline phosphatase (ALP).

FIG. 8 is a series of images of IPS cells. FIG. 8A is a phase contrast image of IPS cells. FIG. 8B-D are images of GFP labeled IPS cells.

FIG. 9 is a series of images showing expression of Oct4, Sox-2 and Nestin in IPS cells. IPS cells from dental pulp expressed the stem cell markers Oct4 and Sox2, as well as the neural precursor marker nestin.

FIG. 10 is a pair of images showing plated IPS cells forming neurospheres.

FIG. 11 is a series of images showing Beta-3-tubulin expression by plated neurospheres. Neurospheres expressed Beta-3-tubulin, a neuronal marker, after neuroinduction.

FIG. 12 is a series of images showing Beta-3-tubulin by single cells. Single cell seeded IPS cells expressed beta-3-tubulin after neuronal induction.

### DETAILED DESCRIPTION

The present invention is based in part on the discovery that a dental stem cell that expresses an Oct3/4 transgene becomes pluripotent. See Example 1.

Thus, in some embodiments, a dental stem cell comprising an Oct3/4 transgene is provided. Oct3/4 (an abbreviation of Octamer3/4) is a homeodomain transcription factor of the POU family. Oct3/4 is also known as POU5F1 (POU class 5 homeobox 1) and Oct-4. The human Oct3/4 amino acid sequence is provided as Genbank Accession No. ABF29403.

As used herein, a stem cell is a relatively undifferentiated cell capable of self-renewal through mitotic cell division and also capable of differentiating into more specialized cell types. As is known in the art, stem cells include embryonic stem cells, which are totipotent, i.e., capable of differentiating into all cell types of the organism from which they were derived, and adult stem cells, which are pluripotent (capable of differentiating into almost all cell types including types from all three germ layers), multipotent (capable of differentiating into several cell types of a closely related family of cells), or unipotent (capable of differentiating into only one type of cell but distinguished from non-stem cells by the ability to self-renew by mitosis).

As used herein, a dental stem cell (DSC; also known as tooth-derived stem cell=TSC) is a stem cell derived from vertebrate tooth pulp. They can be from any tooth of any vertebrate that has teeth. In some embodiments, the dental stem cell is derived from a deciduous tooth. In other embodiments, the dental stem cell is derived from a premolar, a molar, an incisor or a canine.

The Oct3/4 transgene can be expressed under any conditions that can be controlled by control elements operably linked thereto (e.g., promoters, enhancers, etc.) now known or later discovered. In some embodiments, expression of the Oct3/4 protein encoded by the Oct3/4 transgene is inducible. Inducible expression can allow induction of pluripotency when desired; for example, when pluripotency is not wanted until after implantation. In other embodiments, expression of the Oct3/4 protein is constitutive.

In some embodiments, the dental stem cell comprising an Oct3/4 transgene expresses the Oct3/4 protein encoded by the Oct3/4 transgene, and possesses pluripotency. In some of those embodiments, the pluripotent dental stem cell expressing the Oct3/4 protein encoded by the Oct3/4 transgene also comprises a Nanog transgene, a Sox2 transgene or a Lin28 transgene. In some embodiments, the pluripotent dental stem cell comprises an Oct3/4 transgene and a Nanog transgene. Nanog is a transcription factor comprising a conserved homeodomain motif that is localized to the nuclear component of cells. The human Nanog amino acid sequence is provided as Genbank Accession No. AAP49529. In some embodiments, the pluripotent dental stem cell comprises an Oct3/4 transgene and a Sox2 transgene. Sox2, short for SRY (sex determining region Y)-box 2, is a transcription factor involved in the regulation of embryonic development and in the determination of cell fate. The human Sox2 amino acid sequence is provided as Genbank Accession No. AAH13923. In some embodiments, the pluripotent dental stem cell comprises an Oct3/4 transgene and a Lin28 transgene. Lin28 is a cytoplasmic mRNA-binding protein that binds to and enhances the translation of the Igf2 mRNA. The human Lin28 amino acid sequence is provided as Genbank Accession No. AAH28566.

In some embodiments, the Oct3/4-transfected cell further comprises another transgene, such as a Nanog transgene, a Sox2 transgene or a Lin28 transgene. For example, the Oct3/4-transfected cell can further comprise a Nanog transgene. As another example, the Oct3/4-transfected cell can further comprise a Sox2 transgene. As another example, the Oct3/4-transfected cell can further comprise a Lin28 transgene. In other embodiments, the cell comprises a Nanog transgene, a Sox2 transgene and a Lin28 transgene. In additional embodiments, the cell does not comprise a Nanog transgene, a Sox2 transgene or a Lin28 transgene. In some of those embodiments, the cell does not comprise any of a Nanog transgene, a Sox2 transgene or a Lin28 transgene.

The cell can further comprise any other transgene, e.g., a protein or a functional polynucleotide that is expressed by the cell, or a nucleic acid that encodes a functional polynucleotide, e.g., a ribozyme, aptamer or miRNA. The cell may be transfected before, after, or synonymously with the transfection of the cell with the Oct3/4 gene. In some aspects of these embodiments, the cell is transfected with a nucleic acid that encodes a protein, for example a therapeutic protein, such as: a protein missing in the intended recipient of the cell, e.g., a clotting factor, common gamma chain (γ_{c}), or adenosine deaminase; a structural protein, e.g., collagen; an antigen of a disease organism to induce immunity; a growth factor, e.g., to promote the differentiation of the cell (such as transfecting the cell with proinsulin or hepatocyte growth factor to promote production of insulin or differentiation into a pancreatic beta-like cell, or TGF-β3 to promote differentiation into a chondrocyte-like cell); or a protein that provides therapy for a growth factor deficiency (e.g., IL-12) or to fight cancer or infection (e.g., γ-interferon).

In other aspects the nucleic acid encodes a functional polynucleotide. As used herein, a functional polynucleotide is a polynucleotide that has a known function, for example an miRNA, an aptamer, a ribozyme, or an antisense RNA. The functional polynucleotide can promote differentiation of the cell (as in, e.g., Nakajima et al., 2006) or can be utilized for any other purpose, for example, as a cancer therapy (as in, e.g., Saito et al., 2006).

The dental stem cell in these embodiments can be from any species. In some embodiments, the dental stem cell is a mammalian cell, for example a human cell, a rat cell, a rabbit cell, or a mouse cell.

A method of making a pluripotent stem cell is also provided. The method comprises transfecting a dental stem cell with an Oct3/4 gene such that the cell expresses a transgenic Oct3/4 and is pluripotent. In some embodiments of these methods, the cell does not express at least one of a transgenic Nanog, a transgenic Sox2, or a transgenic Lin28. In other embodiments, the cell does not express any of a transgenic Nanog, a transgenic Sox2, or a transgenic Lin28. In additional embodiments, the cell expresses at least one of a transgenic Nanog, a transgenic Sox2, or a transgenic Lin28. In further embodiments, the cell expresses a transgenic Nanog, a transgenic Sox2, and a transgenic Lin28.

These methods can utilize any vector, now known or later discovered, to transfect the cell with the Oct3/4 gene. Nonlimiting examples include naked DNA vectors including plasmids, adenoviral vectors, and lentiviral vectors. In some embodiments, the cell is transfected with a lentivirus comprising the Oct3/4 gene. In some embodiments, the cell is transfected with a lentivirus comprising the Oct3/4 gene and a Sox2 gene.

The pluripotent cell prepared by these methods can be differentiated into any somatic cell desired by, e.g., growing the cells in media known to differentiate pluripotent stem cells into the desired somatic cell. In some embodiments, the method comprises growing the cell in a medium that causes the cell to differentiate into an insulin-secreting cell, a chondrocyte-like cell, a myocyte-like cell, a hair follicle-like cell, or a neuron-like cell. See, e.g., PCT Patent Application PCT/US09/39360, describing methods and media for differentiating pluripotent cells into insulin-secreting cells, chondrocyte-like cells, myocyte-like cells, and hair follicle-like cells. For example, the method can comprise growing the cell in a medium that causes the cell to differentiate into an insulin-secreting cell. As another example, the method can comprise growing the cell in a medium that causes the cell to differentiate into a chondrocyte-like cell. As another example, the method can comprise growing the cell in a medium that causes the cell to differentiate into a myocyte-like cell. As another example, the method can comprise growing the cell in a medium that causes the cell to differentiate into a hair follicle-like cell. As another example, the method can comprise growing the cell in a medium that causes the cell to differentiate into a neuron-like cell.

Also provided herewith is a method of preparing an insulin-secreting cell, for example a pancreatic beta-like cell. The method comprises incubating a pluripotent dental stem cell expressing an Oct3/4 transgene, as described above, in a medium that induces differentiation of a pluripotent cell into an insulin-secreting cell, under conditions such that the dental stem cell differentiates into an insulin-secreting cell. An insulin-secreting cell prepared by this method is also provided.

As used herein, an insulin-secreting cell is a cell that produces insulin. A pancreatic beta-like cell is a cell derived from a stem cell that produces insulin and PDX-1, and/or C-peptide, which are markers characteristic of pancreatic beta cells. Insulin-secreting cells or pancreatic beta-like cells can be used for treatment of type 1 diabetes.

Any medium known to differentiate a stem cell into an insulin-producing cell or a pancreatic beta-like cell can be used in these methods. See, e.g., PCT Patent Application PCT/US09/39360. In some embodiments, the medium for these methods comprises activin, exendin, pentagastrin, hepatocyte growth factor, and/or noggin. In certain specific embodiments, the medium comprises 0.001-1000 nM activin A, 0.001-1000 nM extendin-4 and 0.001-1000 nM pentagastrin. In more specific embodiments, the medium comprises 0.5-10 nM activin-A, 2-30 nM exendin-4, 2-30 nM pentagastrin and 20-300 pM. In additional embodiments, the medium comprises low glucose DMEM supplemented with about 10 mM nicotinamide, about 2 nM activin-A, about 10 nM exendin-4, about 100 pM hepatocyte growth factor, about 10 nM pentagastrin, B-27 supplement, N-2 Supplement, and at least one antibiotic. Where the medium comprises noggin, that compound is generally added to a concentration of about 100-1000 ng/ml, more specifically about 400 ng/ml.

In some aspects, the method further comprises testing the cell for a characteristic of a pancreatic beta cell. Any such characteristic can be tested in this method. In some embodiments, the characteristic is the secretion of insulin. Another characteristic that can be tested is the production of PDX1. A further characteristic that can be tested is the production of C-peptide. In further embodiments the cells are tested for all three characteristics. These characteristics can be tested by any means known in the art. In some embodiments, they are tested by ELISA or fluorescent antibody cell staining.

In additional embodiments, a method of preparing a chondrocyte-like cell is provided. The method comprises incubating a pluripotent dental stem cell expressing an Oct3/4 transgene in a medium that induces differentiation of a pluripotent cell into a chondrocyte-like cell, under conditions such that the dental stem cell differentiates into a chondrocyte-like cell. A chondrocyte-like cell prepared by this method is also provided.

As used herein, a chondrocyte-like cell is a cell derived from a stem cell that stains with safranin 0, and/or comprises glycosaminoglycans. Chondrocyte-like cells can be used for the treatment of arthritis or for augmentative or reconstructive surgery.

Any medium known to differentiate stem cells into chondrocyte-like cells can be used in these methods. See, e.g., PCT Patent Application PCT/US09/39360. In some embodiments, the medium comprises TGF-β3.

In various aspects, the method further comprises testing the cell for a characteristic of a chondrocyte. Any characteristic that distinguishes a chondrocyte from other cells can be tested. In some embodiments, the cell is tested for safranin O staining and/or glycosaminoglycan content.

In further embodiments, a method of preparing a myocyte-like cell is provided. The method comprises incubating a pluripotent dental stem cell expressing an Oct3/4 transgene in a medium that induces differentiation of a pluripotent cell into a myocyte-like cell, under conditions such that the dental stem cell differentiates into a myocyte-like cell. A myocyte-like cell prepared by this method is also provided.

As used herein, a myocyte-like cell is a cell derived from a stem cell that comprises myoD, myf5, desmin and/or myosin. Myocyte-like cells can be used to treat muscular dystrophy, atrophy, or for the enhancement of muscle strength.

Any medium that induces the differentiation of a stem cell into a myocyte-like cell can be used for these methods. In some embodiments, the medium comprises dexamethasone and hydrocortisone.

In some aspects, the method further comprises testing the cell for a characteristic of a myocyte. Any characteristic that distinguishes a myocyte from other cells can be tested. In some embodiments, the cell is tested for myoD, myf5, desmin and/or myosin, by any means known in the art.

In additional embodiments, a method of preparing a hair follicle-like cell is provided. The method comprises incubating a pluripotent dental stem cell expressing an Oct3/4 transgene in a medium that induces differentiation of a pluripotent cell into a hair follicle-like cell, under conditions such that the dental stem cell differentiates into a hair follicle-like cell. Examples of such media include dermal papilla media or outer root sheath media. A hair follicle-like cell prepared by this method is also provided.

As used herein, a hair follicle-like cell is a cell derived from a stem cell that exhibits a characteristic of a hair follicle. Examples of such characteristics are the presence of CD44, Lef1, CD59 and/or CK14. Hair follicle-like cells can be used for hair follicle regeneration in the treatment of alopecia or baldness.

In various aspects, these methods further comprise testing the cell for a characteristic of a hair follicle. Any characteristic that distinguishes a hair follicle cell from other cells can be tested, by any means known in the art. Examples include CD44, Lef1, CD59 and/or CK14.

In further embodiments, a method of preparing a neuron-like cell is provided. In some embodiments, the method comprises incubating a pluripotent dental stem cell expressing an Oct3/4 transgene and a Sox2 transgene in a medium that induces differentiation of a pluripotent cell into a neuron-like cell, under conditions such that the dental stem cell differentiates into a neuron-like cell. For example, a pluripotent dental stem cell expressing an Oct4 transgene and a Sox2 transgene can be incubated in a medium that induces differentiation of a pluripotent cell into a neuron-like cell, under conditions such that the dental stem cell differentiates into a neuron-like cell. A neuron-like cell prepared by this method is also provided.

As used herein, a neuron-like cell is a cell derived from a stem cell that exhibits a characteristic of a neuron cell. In some embodiments, a neuron-like cell can express a marker characteristic of neuron cells. For example, a neuron-like cell can express nestin. As another example, a neuron-like cell can express beta-III-tubulin. In some embodiments, a neuron-like cell can exhibit characteristics of cells including, but not limited to, basket cells, betz cells, medium spiny neurons, purkinje cells, pyramidal cells, renshaw cells, granule cells, or anterior horn cells. In some embodiments, a neuron-like cell can exhibit characteristics of neuron cells including, but not limited to, afferent neurons, efferent neurons, or interneurons. In some embodiments, a neuron-like cell can exhibit characteristics of neuron cells including, but not limited to, cholinergic neurons, GABAergic neurons, glutamatergic neurons, dopaminergic neurons, or serotonergic neurons. In some embodiments, a neuron-like cell can produce a neurotransmitter characteristic of a neuron cell. For example, a neuron-like cell can produce a neurotransmitter including, but not limited to, acetylcholine, gamma aminobutyric acid, glutamate, dopamine, or serotonin. In some embodiments, a neuron-like cell demonstartes neuron cell characteristics according to a Neural Colony-Forming Cell (NCFC) Assay (see e.g., Louis et al., 2008, “Enumeration of neural stem and progenitor cells in the neural colony-forming cell assay”, Stem Cells 26(4) 988-996).

Neuron-like cells can be used to treat, for example, a neurodegenerative disease, such as Parkinson's disease, Alzheimer's disease, Charcot-Marie-Tooth disease, or Myasthenia Gravis, and neuro-tissue injuries, such as spinal cord injuries.

Any medium that induces the differentiation of a stem cell into a neuron-like cell can be used for these methods. See e.g., Roy et al., 2000, “In vitro neurogenesis by progenitor cells isolated from the adult human hippocampus”, Nature Medicine 6(3) 271-277; Taupin et al., 2000, “FGF-2-responsive neural stem cell proliferation requires CCg, a novel autocrine/paracrine cofactor”, Neuron 28(2) 385-397; Reynolds and Weiss, 1992, “Generation of neurons and astrocytes from isolated cells of the adult mammalian central nervous system” Science 255(5052) 1707-1710. In some embodiments, the medium for differentiation of a stem cell into a neuron-like cell comprises Epidermal growth factor (EGF). In some embodiments, the medium for differentiation of a stem cell into a neuron-like cell comprises fibroblast growth factor (FGF). In some embodiments, the medium for differentiation of a stem cell into a neuron-like cell comprises Epidermal growth factor (EGF) and fibroblast growth factor (FGF). In some embodiments, the medium for differentiation of a stem cell into a neuron-like cell comprises CCg.

In some aspects, the method further comprises testing the cell for a characteristic of a neuron. Any characteristic that distinguishes a neuron from other cells can be tested. In some embodiments, the cell is tested for nestin or beta-III-tubulin, by any means known in the art (see e.g., Example 2). In some embodiments, cell are tested for an ability to form neurospheres, by any means known in the art (see e.g., Example 2). In some embodiments, cell are tested according to a Neural Colony-Forming Cell (NCFC) Assay (see e.g., Louis et al., 2008, “Enumeration of neural stem and progenitor cells in the neural colony-forming cell assay”, Stem Cells 26(4) 988-996.

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment (especially in the context of certain of the following claims) can be construed to cover both the singular and the plural. Additionally, the use of “or” is intended to include “and/or”, unless the context clearly indicates otherwise. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation or discussion of a reference herein shall not be construed as an admission that such is prior art to the present invention. Applicants reserve the right to challenge the accuracy and pertinence of a cited reference.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Transformation with Oct3/4 is Sufficient to Reprogram Dental Stem Cells into Pluripotency

Exfoliating deciduous incisors and permanent third molars of multiple donors were collected with Columbia University Institutional Review Board approval. The dental pulps were isolated and enzyme-digested. Mononucleated and adherent cells were cultured in DMEM-LG medium containing 10% FBS and 1% antibiotics in 10 cm cell culture dishes. Single cells in suspension were then isolated from heterogeneous dental stem cells and cultured under the same conditions for 2 weeks. Following this, the monoclonal cells were transferred to 6-well culture plates. The isolated cells were cultured in DMEM containing 10% fetal bovine serum (FBS).

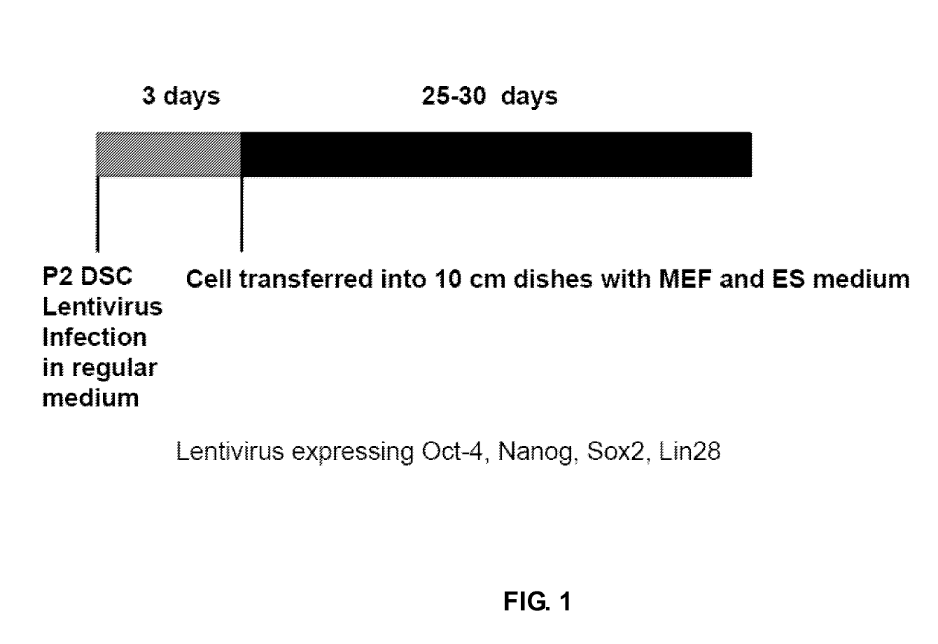

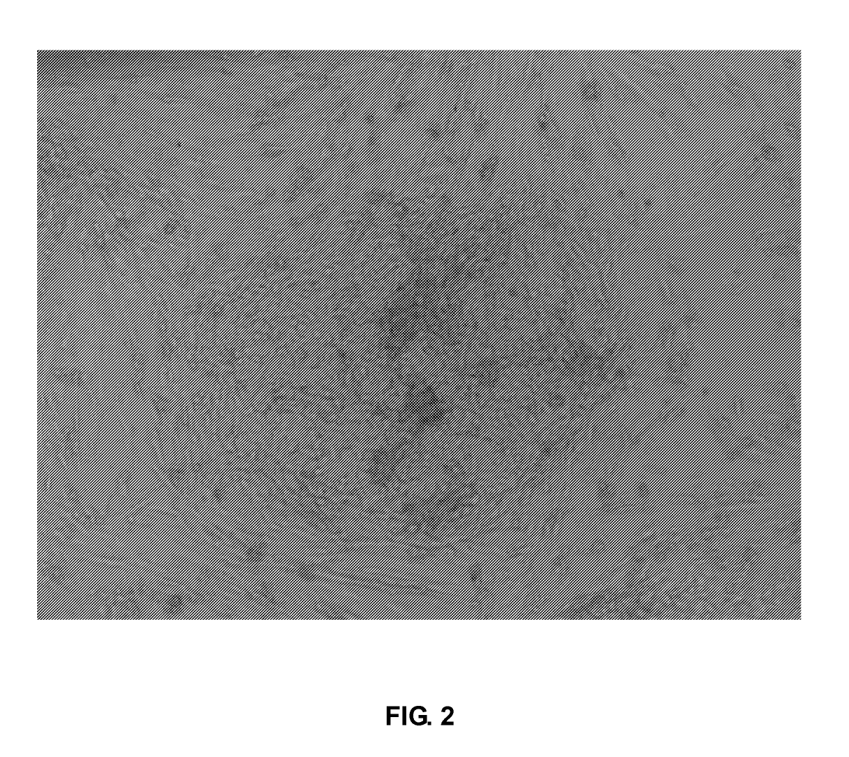

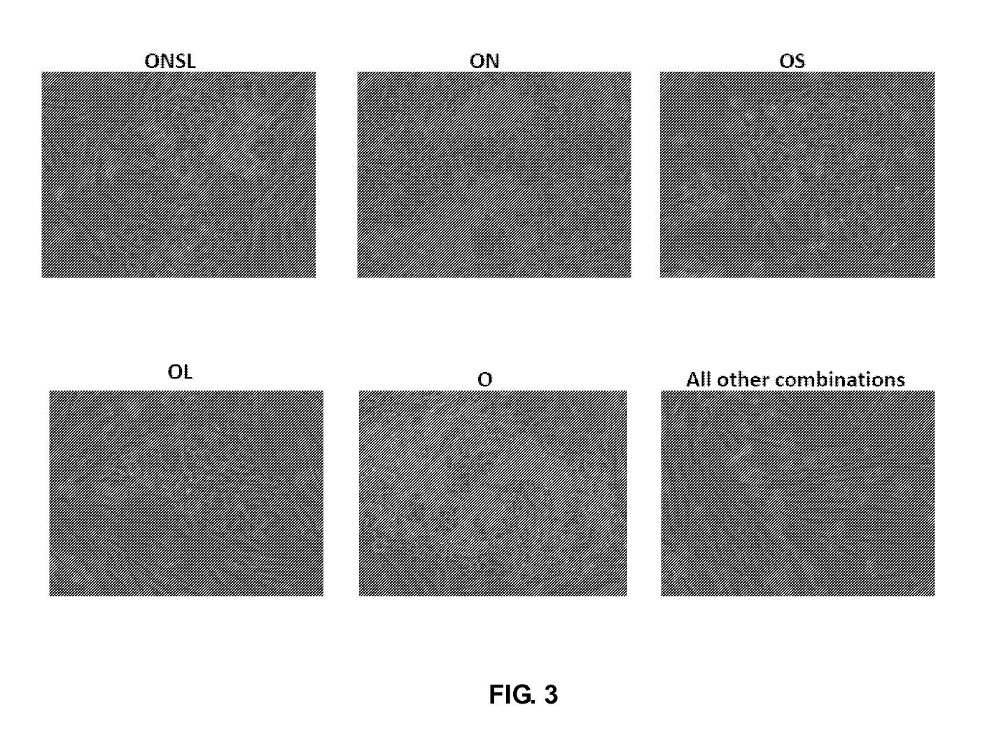

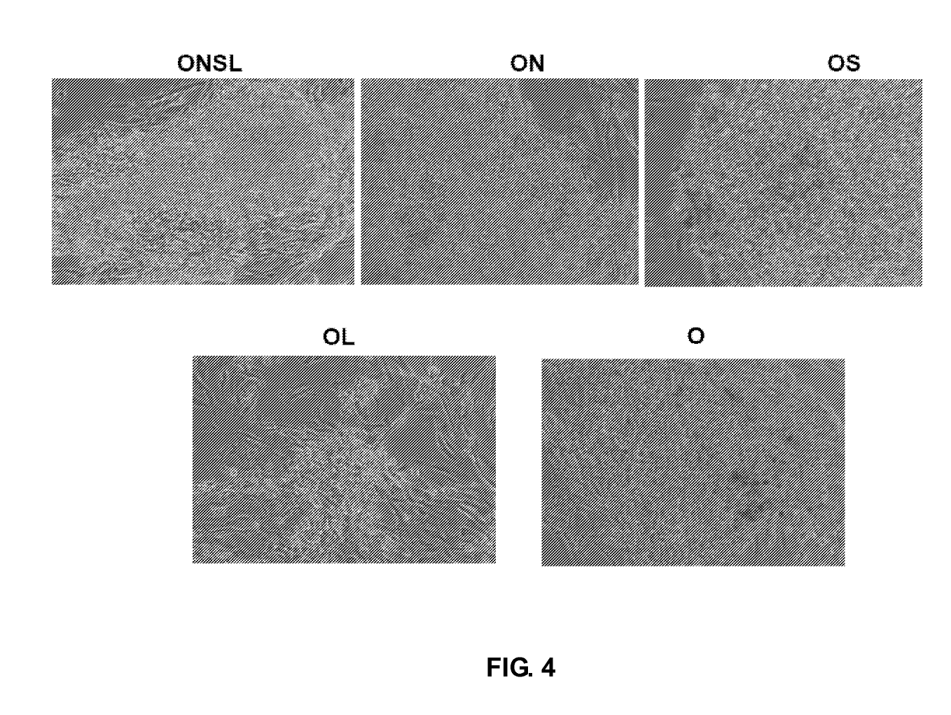

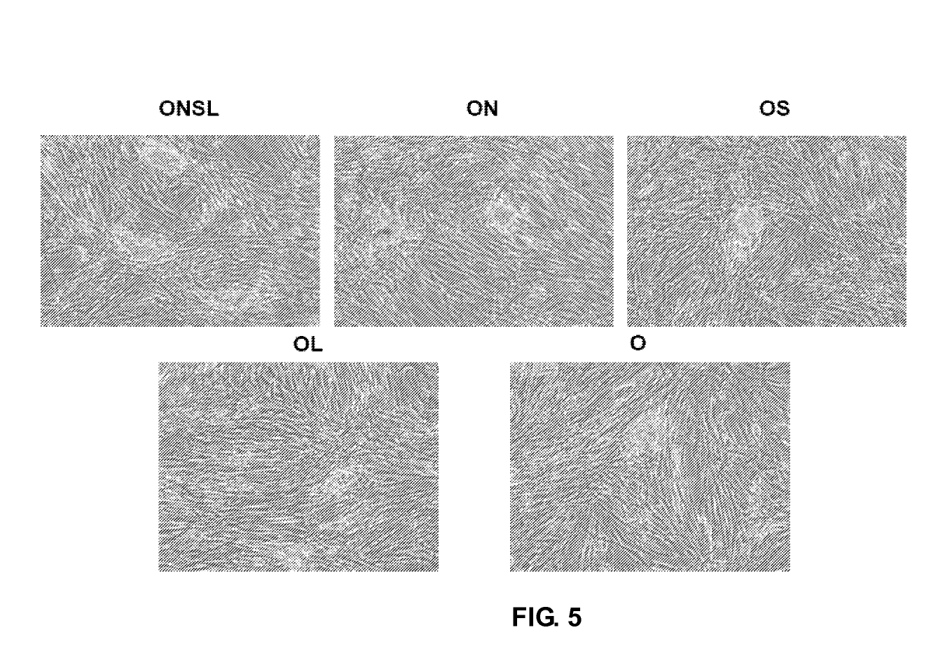

The protocol for these experiments is provided in FIG. 1 as a timeline. Passage 2 cells were used for reprogramming. The cells were seeded in 6 well plates (3×10⁵/well) and then infected with lentiviruses (Stemgent) expressing Sox-2, Oct3/4, Nanog and/or Lin-28 (described as S, O, N, L in the Figures) individually or in various combinations of 4 factors (S, O, N, L), 2 factors (SO, SN, SL, ON, OL and NL) or a single factor (S, O, N, or L). Three days after transfection, the cells were trypsinized and transferred into the 10 cm gelatin treated dishes with Mouse Embryonic Fibroblast (MEF) as a feeder. One day later, after the cells attached to dishes, the cell culture medium was changed into ES cell medium (DMEM/F-12, 20% knockout serum replacement, 2 mM L-glutamine, nonessential amino acids, 1 mM 2-mecaptoethanol and 100 ng bFGF). Twelve days after culturing in ES cell medium, the cells infected with Oct-3/4, with or without other factor(s), form typical granulated ES cell-like colonies (FIG. 3). Compare with FIG. 2, showing an iPS colony. No such colony morphology was observed with other combinations (FIG. 3). At week 3, the ES cell-like clones were picked manually under the stereomicroscope. FIG. 4 shows the colonies three weeks after transfection. The colonies continued to expand, still showing a growth pattern typical of iPS colonies. One clone picked from the Oct3/4-transfected colonies was amplified on the feeder layer. After 2 weeks culture, the cells maintained the ES cell morphology (FIG. 5, 6).

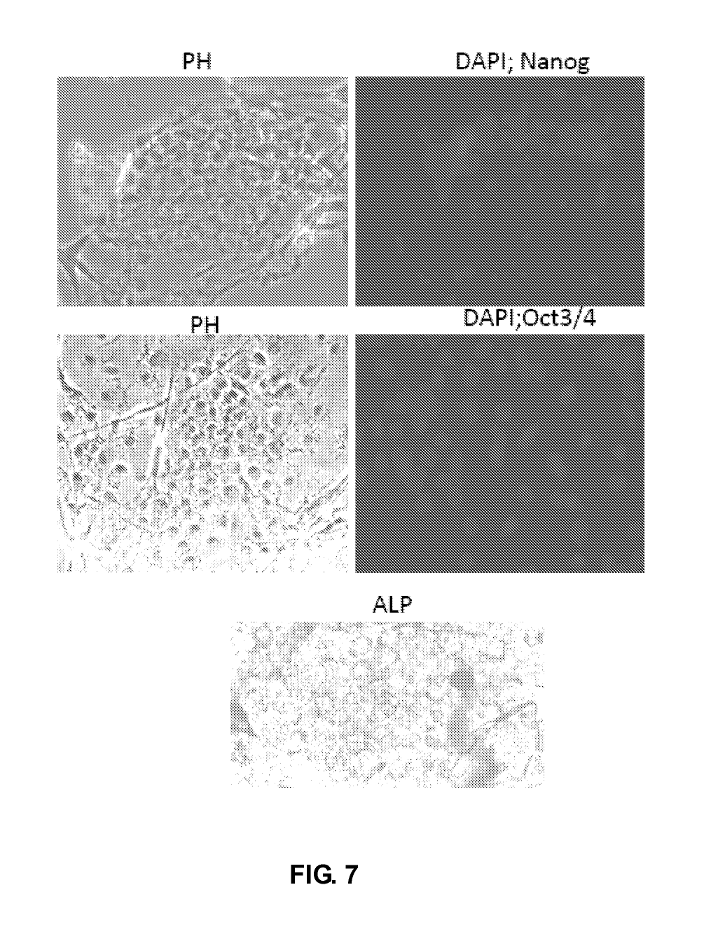

The colonies were tested for Nanog and Oct3/4 (FIG. 7). They were first fixed with formalin for 10 min and then treated with PBS containing 0.1% Triton. The cells were then incubated with blocking buffer for 30 min and then Oct3/4 or Nanog antibodies. Rhodamine conjugated secondary antibodies were used to detect the signal. The cells were Nanog and Oct3/4 positive (FIG. 7). The presence of alkaline phosphatase (ALP) was further tested stained for alkaline phosphatase (ALP) activity according to the protocol described in Moioli et al., 2008. in these colonies, since embryonic stem cells are characteristically ALP-positive. The Oct3/4-transfected cells were ALP-positive (FIG. 7) further establishing the embryonic stem cell-like nature of those cells.

### Example 2

### Differentiation of Neural-Like Cell from Dental Stem Cell Transfected with Sox-2 and Oct/4

Dental pulp stem cells were infected with GFP labeled lentiviruses expressing Sox-2 and Oct/4+. Transfected cells were differentiated in vitro in chemically defined medium to form neural-like cells. Immunocytochemistry was used to confirm neural differentiation.

Reprogramming

Isolated dental pulp stem cells cultured in DMEM with 10% FBS were infected with lentiviruses (Stemgent) expressing Oct3/4 and c-Myc with the MOI (10:1) in presence of 5 μg/ml polybrene. Two days after infection, the cells (about 10000 cells) were trypsinized and transferred into the Gelatin (0.1% in water) coated 10 cm dishes seeded with 1.5 million mitomycin C treated mouse embryonic fibroblasts. The media were changed to DMEM/F12 with 20% Knockout Serum Replacement. Two weeks later, the colonies were observed and picked manually. The colonies were further expanded in the DMEM/F12 with 10% FBS and 1 ng/ml bFGF.

Cell Assays

Eight chambered well slides (BD Falcon) or 24 well plates (BD Falcon) were coated first with polyornithine (Sigma) overnight in room temperature, and then washed with distilled deionized water (Cellgro). Another coating was performed with laminin (2 ug/cm²) overnight in an incubator and then washed once with distilled deionized water, to let dry for 2 hours. The IPS cells were seeded at a density of 5000 cells/cm² in a mixture consisting of Neurobasal-A medium, B27 supplement (Gibco), Glutamax-I (Gibco), 20 ng/ml human FGF-basic (Peprotech), 10 ng/ml EGF (Peprotech) and antibiotic-antimycotic (10,000 U/ml penicillin, 10,000 μg/ml streptomycin, Atlanta biologicals). The cells were maintained in an incubator with 95% CO₂ with change of medium every other day.

Immunocytochemistry

After 14 days of neural induction cells were stained for beta-3-tubulin marker. Cells were washed with PBS (BioWhittaker) and fixed with 10% formalin (Fisher Scientific) for 5 minutes. 0.1% Triton PBS wash for 15 minutes was performed, followed by 2×PBS wash. Then blocking buffer (Odyssey) was added for a period of 30 minutes on a gentle shaker. TUJ-1 (Beta-3-tubulin) antibody was added (Santa Cruz Biotechnology, 1:100), and incubated in room temperature for 1 hour on a shaker. 10 minute 0.1% PBS-tween PBS wash and then 2×PBS wash. Secondary antibody, Alexa Fluor 488 goat-anti-mouse Ig-G (1:1000, Invitrogen) was added for 1 hour in room temperature followed by a 3×PBS wash. Subsequently, DAPI solution was added for 20 minutes, followed by PBS for storage.

GFP Labeling

Seeding density of 2500 cells/cm² to 24 wells in a medium mixture consisting of DMEM/F12+Glutamax-I (Gibco), 10% FBS (Gibco), 1 ng/ml human FGF-basic (Peprotech) and antibiotic-antimycotic (10,000 U/ml penicillin, 10,000 μg/ml streptomycin, Atlanta biologicals) was performed. Next day cells were infected with 200 μl GFP Lentivirus (Cellbiolabs) for 72 hours. Medium change occurred every other day and expansion of cell number occurred until there was approximately 2×10⁶ cells for FACS of GFP labeled cells.

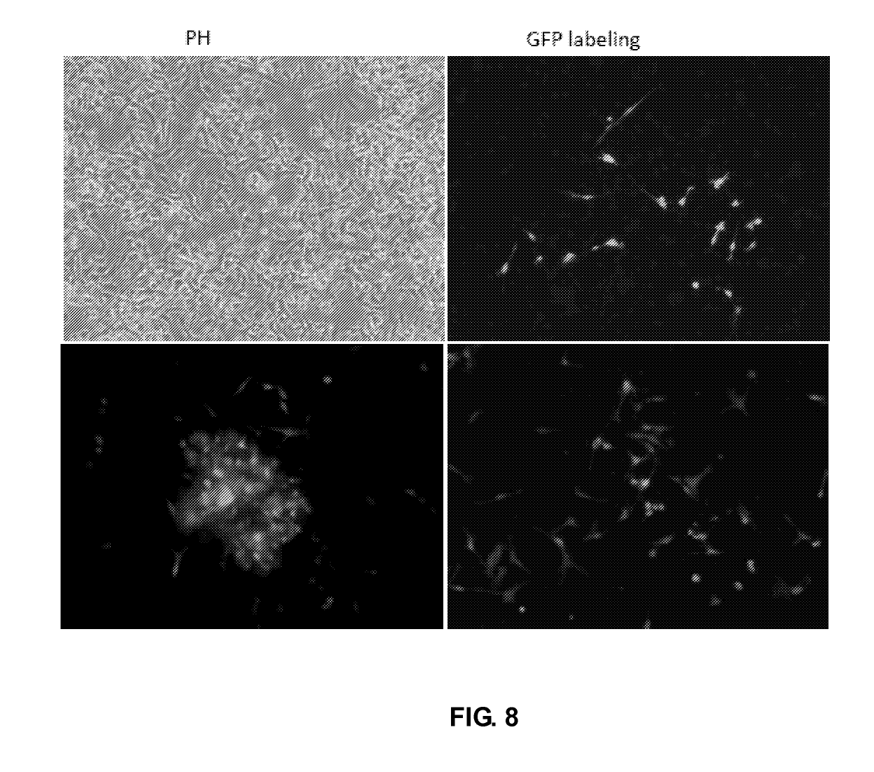

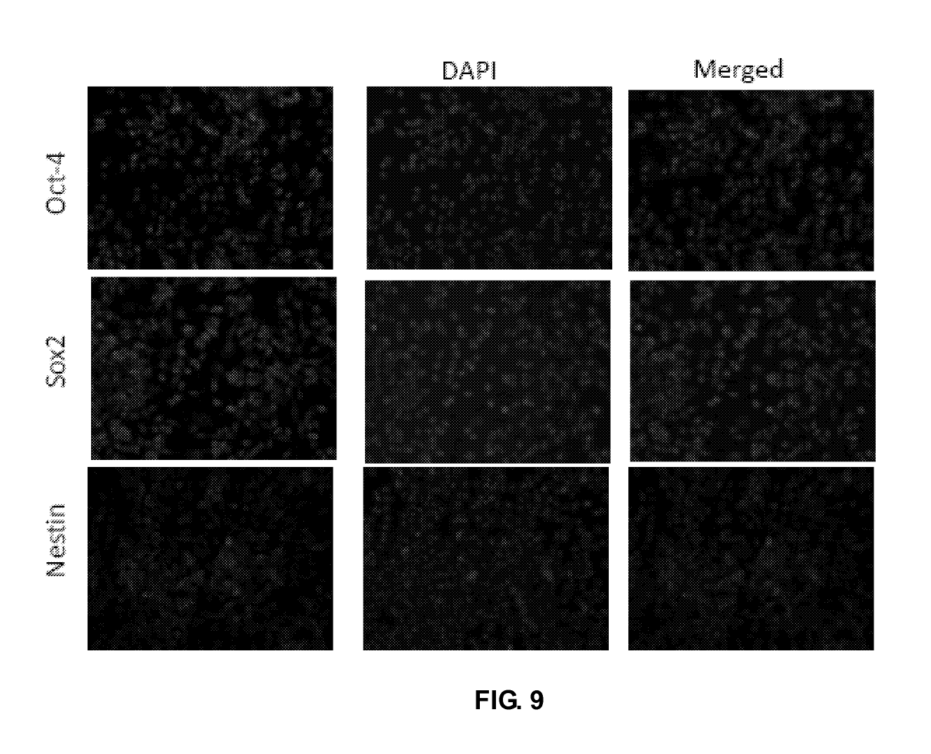

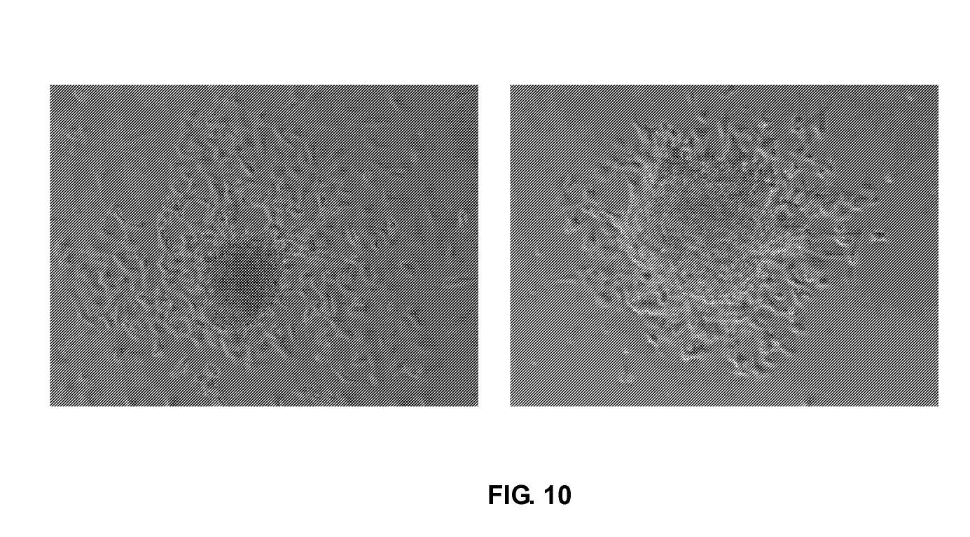

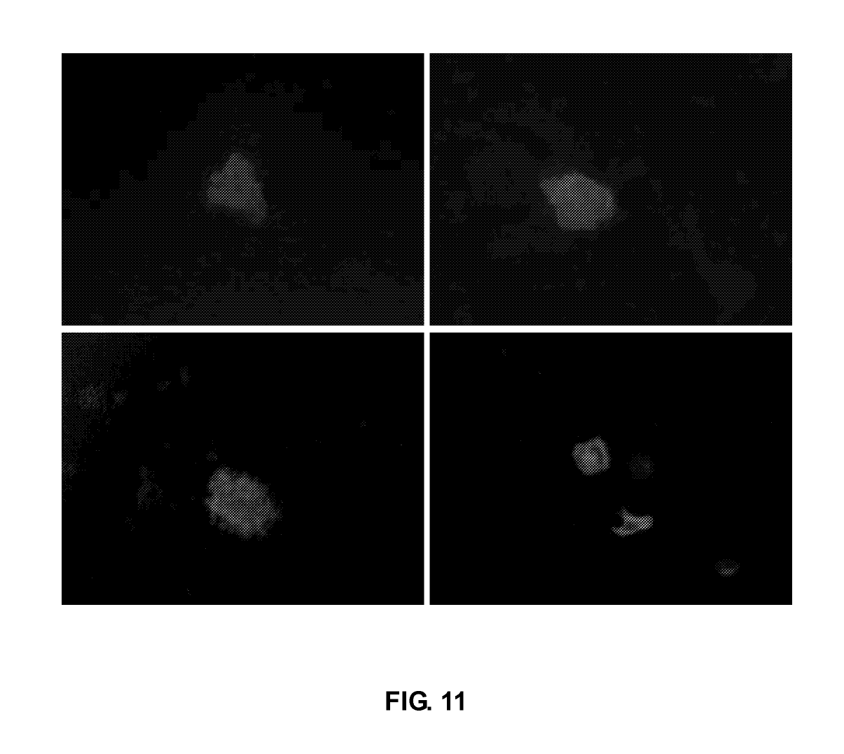

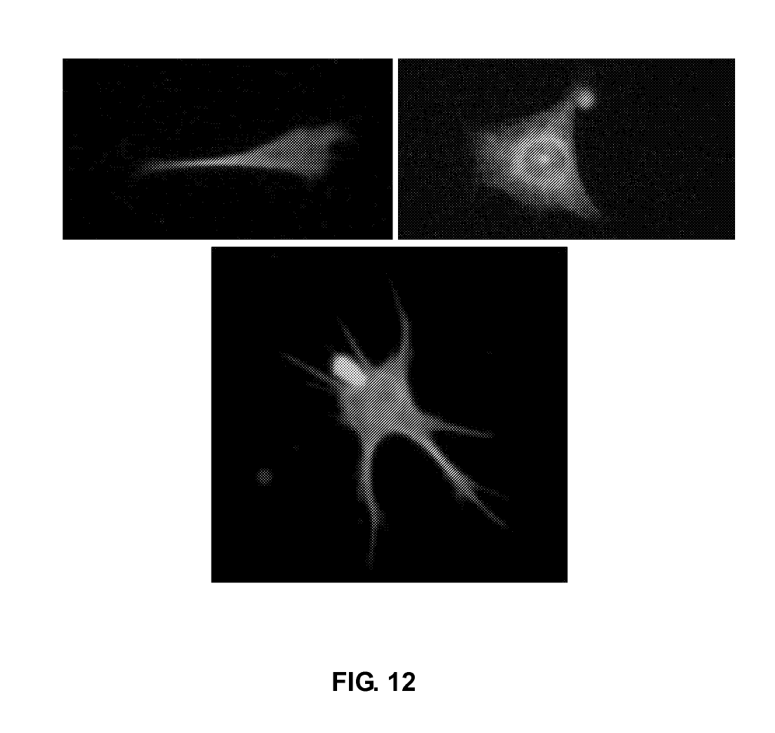

Results showed that IPS cells expressed GFP (see e.g., FIG. 8). The IPS cells from dental pulp were also shown to express the stem cell markers Oct4 and Sox2, as well as the neural precursor marker nestin (see e.g., FIG. 9). IPS cells were also shown to be capable forming neurospheres (see e.g., FIG. 10). Also shown was that plated neurospheres expressed Beta-3-tubulin, a neuronal marker, after neuroinduction (see e.g., FIG. 11). Similarly, single cell seeded IPS cells were shown to express beta-3-tubulin after neuronal induction (see e.g., FIG. 12).

### REFERENCES

Alhadlaq A, Mao J J 2004 Mesenchymal stem cells: isolation and therapeutics. Stem Cells Dev 13:436-448.
D'Amour K A et al. 2006 Production of pancreatic hormone-expressing endocrine cells from human embryonic stem cells. Nat Biotechnol 24:1392-1401.
Kim J B et al. 2009 Oct3/4-Induced Pluripotency in Adult Neural Stem Cells. Cell 136:411-419.
Liu T M et al. 2008 Effects of ectopic Nanog and Oct3/4 overexpression on mesenchymal stem cells.
Louis et al. 2008 Enumeration of neural stem and progenitor cells in the neural colony-forming cell assay, Stem Cells 26(4) 988-996.
Lumelsky N, Blondel O, Laeng P, Velasco I, Ravin R, McKay R 2001 Differentiation of embryonic stem cells to insulin-secreting structures similar to pancreatic islets. Science 292:1389-1394.
Mao J J et al. 2006 J Dent Res 85:966-979.
Marion N R, Mao J J 2006 Mesenchymal stem cells and tissue engineering. Methods Enzymol 420:339-361.
Miura M et al. 2003 Proc. Natl. Acad. Sci. USA 100:5807-5812.
Moioli E K, Clark P A, Chen M, Dennis J E, Erickson H P, Gerson S L, Mao J J 2008 Synergistic actions of hematopoietic and mesenchymal stem/progenitor cells in vascularizing bioengineered tissues. PLoS ONE 3:e3922.
Morsczeck et al. 2005 Matrix Biol 24:155-165.
Mueller T et al. 2009 Analysis of OCT3/4 expression in an extended panel of human tumor cell lines from multiple entities and in human mesenchymal stem cells. Cell Mol. Life. Sci. 66:495-503.
Nakajima N et al. 2006 Biochem Biophys Res Comm 350:1006-1012.
Peptan I A et al. 2006 Plast Reconstr Surg 117:1462-1470.
Reynolds and Weiss 1992 Generation of neurons and astrocytes from isolated cells of the adult mammalian central nervous system, Science 255(5052) 1707-1710.
Reynolds et al. 2004 Differentiation 72:566-575.
Roy et al. 2000 In vitro neurogenesis by progenitor cells isolated from the adult human hippocampus, Nature Medicine 6(3) 271-277.
Saito et al. 2006 Cancer Cell 9:435-443.
Takahashi K, Yamanaka S 2006 Induction of pluripotent stem cells from mouse embryonic and adult fibroblast cultures by defined factors. Cell 126:663-676.
Taupin et al. 2000 FGF-2-responsive neural stem cell proliferation requires CCg, a novel autocrine/paracrine cofactor, Neuron 28(2) 385-397.
Timper et al. 2006 Biochem Biophys Res Comm 341:1135-1140.
Yen and Sharpe 2007 Cell Tissue Res DOI 10.1007/s00441-007-0467-6 (review)
Yu J et al. 2007 Induced pluripotent stem cell lines derived from human somatic cells. Science 21:1917-1920.
U.S. Pat. No. 6,767,740 B2.
U.S. Pat. No. 7,052,907 B2.
U.S. Pat. No. 7,326,572 B2.
U.S. Patent Application Publication US070274958 A1.
U.S. Patent Application Publication US080038820 A1.
U.S. Patent Application Publication US080248005 A1.
U.S. Patent Application Publication US090047263 A1.
PCT Patent Publication WO0207679A2.
PCT Patent Publication WO03066840 A2.
PCT Patent Publication WO04073633 A2.
PCT Patent Publication WO06010600A2.
PCT Patent Publication WO07014639 A2.
PCT Patent Application PCT/US09/39360.

1-46. (canceled)

47. A dental stem cell comprising an Oct3/4 transgene.

48. The cell of claim 47, wherein the cell
expresses an Oct3/4 protein encoded by the Oct3/4 transgene and possesses pluripotency.

49. The cell of claim 48, comprising at least one feature selected from the group consisting of:
the cell also comprises a Nanog transgene, a Sox2 transgene or a Lin28 transgene;
the cell also comprises a Sox2 transgene, wherein the cell expresses a Sox2 protein encoded by the Sox2 transgene;
the cell also comprises a Nanog transgene, a Sox2 transgene and a Lin28 transgene;
the cell does not comprise at least one of a Nanog transgene, a Sox2 transgene or a Lin28 transgene; and
the cell does not comprise any of a Nanog transgene, a Sox2 transgene or a Lin28 transgene.

50. The dental stem cell of claim 48, wherein the cell is a mammalian cell or a human cell.

51. A method of making a pluripotent stem cell, the method comprising transfecting a dental stem cell with an Oct3/4 gene such that the cell expresses a transgenic Oct3/4 and is pluripotent.

52. The method of claim 51, comprising at least one feature selected from the group consisting of:
the cell does not express at least one of a transgenic Nanog, a transgenic Sox2, or a transgenic Lin28;
the cell does not express any of a transgenic Nanog, a transgenic Sox2, or a transgenic Lin28;
the cell expresses at least one of a transgenic Nanog, a transgenic Sox2, or a transgenic Lin28;
the cell expresses a transgenic Nanog, a transgenic Sox2, and a transgenic Lin28;
the cell is transfected with a lentivirus comprising the Oct3/4 gene; and
the method comprises growing the cell in a medium that causes the cell to differentiate into an insulin-secreting cell, a chondrocyte-like cell, a myocyte-like cell, a hair follicle-like cell, or a neuron-like cell.

53. A method of differentiating the cell of claim 48, comprising:
incubating the dental stem cell of claim 48 in a medium that induces differentiation of a pluripotent cell into a differentiated cell;
wherein the method comprises at least one feature selected from the group consisting of:
(i) the differentiated cell is an insulin-secreting cell and the method comprises incubating the dental stem cell of claim 48 in a medium that induces differentiation of a pluripotent cell into an insulin-secreting cell, under conditions such that the dental stem cell differentiates into an insulin-secreting cell;
(ii) the differentiated cell is a chondrocyte-like cell and the method comprises incubating the dental stem cell of claim 48 in a medium that induces differentiation of a pluripotent cell into a chondrocyte-like cell, under conditions such that the dental stem cell differentiates into a chondrocyte-like cell;
(iii) the differentiated cell is a myocyte-like cell and the method comprises incubating the dental stem cell of claim 48 in a medium that induces differentiation of a pluripotent cell into a myocyte-like cell, under conditions such that the dental stem cell differentiates into a myocyte-like cell;
(iv) the differentiated cell is a hair follicle-like cell and the method comprises incubating the dental stem cell of claim 48 in a medium that induces differentiation of a pluripotent cell into a hair follicle-like cell, under conditions such that the dental stem cell differentiates into a hair follicle-like cell;
(vi) the differentiated cell is a neuron-like cell and the method comprises incubating the dental stem cell of claim 48, further comprising a Sox2 transgene, in a medium that induces differentiation of a pluripotent cell into a neuron-like cell, under conditions such that the dental stem cell differentiates into a neuron-like cell.

54. The method of claim 53, the differentiated cell is an insulin-secreting cell and the method comprises at least one feature selected from the group consisting of:
the medium comprises activin, extendin, pentagastrin, and hepatocyte growth factor;
the medium comprises noggin;
the method further comprises testing the cell for a characteristic of an insulin-secreting cell or a pancreatic beta cell;
the method further comprises testing the cell for a characteristic of an insulin-secreting cell or a pancreatic beta cell, wherein the cell is tested for secretion of insulin;
the method further comprises testing the cell for a characteristic of an insulin-secreting cell or a pancreatic beta cell, wherein the cell is tested for PDX1; and
the method further comprises testing the cell for a characteristic of an insulin-secreting cell or a pancreatic beta cell, wherein the cell is tested for C-peptide.

55. The method of claim 53, wherein the differentiated cell is a chondrocyte-like cell and the method comprises at least one feature selected from the group consisting of:
the medium comprises TGF-β3;
the method further comprises testing the cell for a characteristic of a chondrocyte; and
the method further comprises testing the cell for a characteristic of a chondrocyte, wherein the cell is tested for safranin O staining or glycosaminoglycan content.

56. The method of claim 53, wherein the differentiated cell is a myocyte-like cell and the method comprises at least one feature selected from the group consisting of:
the medium comprises dexamethasone and hydrocortisone;
the method further comprising testing the cell for a characteristic of a myocyte; and
the method further comprising testing the cell for a characteristic of a myocyte, wherein the cell is tested for myoD, myf5, desmin or myosin.

57. The method of claim 53, wherein the differentiated cell is a hair follicle-like cell and the method comprises at least one feature selected from the group consisting of:
the medium comprises dermal papilla media or outer root sheath media;
the method further comprises testing the cell for a characteristic of a hair follicle; and
the method further comprises testing the cell for a characteristic of a hair follicle, wherein the cell is tested for CD44, Lef1, CD59 or CK14.

58. The method of claim 53, wherein the differentiated cell is a neuron-like cell and the method comprises at least one feature selected from the group consisting of:
the medium comprises Epidermal growth factor (EGF), fibroblast growth factor (FGF), or both;
the method further comprises testing the cell for a characteristic of a neuron-like cell;
the method further comprises testing the cell for a characteristic of a neuron-like cell, wherein the cell is tested for expression of nestin or beta-III-tubulin.

59. The method of clam 53, wherein the cell is a human cell.

60. A cell prepared by the method of claim 53, wherein the cell is a differentiated cell selected from the group consisting of: an insulin-secreting cell, a chondrocyte-like cell, a myocyte-like cell, a hair follicle-like cell, and a neuron-like cell.

61. The cell of claim 60, wherein the differentiated cell is a human cell.
